(19) United States
(12) Patent Application Publication
(10) Pub. No.: US 2026/0344691 A1
(43) Pub. Date: Nov. 5, 2026

(54) FOODSTUFF

(71) Applicant: MARLOW FOODS LIMITED, Stokesley (GB)

(72) Inventors: Anna Jayne TAYLOR, Stokesley (GB); Linda Christina PRAVINATA, Stokesley (GB); Albertus Antonius Gerardus HAFKAMP, Stokesley (GB); Willem Cornelis MEIJER, Stokesley (GB)

(21) Appl. No.: 19/487,328

(22) PCT Filed: May 7, 2024
(86) PCT No.: PCT/GB2024/051183

§ 371 Date: Nov. 24, 2025

(30) Foreign Application Priority Data
May 25, 2023 (GB) 2307835.5

(51) Int. Cl. A23C 20/02; A23L 31/00

(52) U.S. Cl. CPC A23C 20/02; A23L 31/00

(57) ABSTRACT

A foodstuff, for example a hard, vegan cheese, comprises a filamentous fungus, for example Fusarium venenatum and a vegetable oil with a melting point of at least 20 °C, wherein the foodstuff has a hardness of at least 1000 g, for example in the range 12000 to 40000 g.

This invention relates to a foodstuff and particularly, although not exclusively, relates to a cheese-like foodstuff which includes no animal products and is therefore suitable for vegans. Such a foodstuff may be referred to herein as aforesaid or as a “vegan cheese”, a vegan cheese-like foodstuff (or similar term). Especially preferred embodiments relate to a vegan cheese which is similar in a range of properties to semi-hard or hard, milk-based cheeses.

Vegan cheeses are well known in general, and they may be made from a range of ingredients. For example, the main ingredients in vegan cheeses may be nuts, soy milk, and soy yogurt and common plant-based proteins or vegetable proteins used in vegan cheeses are derived from edible sources of protein, such as soybeans, almonds, and their milk.

Many vegan cheeses are commercially available. For example, a company called Honestly Tasty sells a range of vegan cheeses. One cheese includes the following ingredients: Cashews, Coconut Oil, White Miso (Soy), Nutritional Yeast, Onion, Salt, Mustard. Another includes the following ingredients: Shea Butter, Rice Bran Oil, almonds, Tapioca Flour, Miso (soy), Salt, Nutritional Yeast, Carrageenan, White Truffle Flavoured Olive Oil. Another includes the following ingredients: Coconut Oil, almonds, Tapioca Flour, Salt, Nutritional Yeast, Carrageenan, Agar, Lactic Acid.

Cheeses are sold under the Trade Mark VIOLIFE. A “mature cheddar” from the company includes the following ingredients: Water, Coconut Oil, Starch, Modified Starch, Sea Salt, Cheddar Flavour, Olive Extract, Colour: (Paprika Extract, B-Carotene), Vitamin B12. A grated cheese is stated to include the following ingredients: Water, Coconut Oil (24%), Modified Starch, Starch, Sea Salt, Flavourings, Olive Extract, Colour: B-Carotene, Vitamin B12. A “Parmesan Cheese” is stated to include the following ingredients: coconut oil, rice protein, potato & rice starch, modified food starch (potato), olive extract, vitamin B12.

Cheeses sold under the brand NURISHH are said to include the following ingredients: Water, Modified Food Starch, Coconut Oil, Calcium Citrate, Salt, No More Than 2% of Each: Natural Flavor, Citric Acid, Color (Beta-Carotene, Paprika Extract), Sugar, Cyanocobalamin (Vitamin B12).

Freshbrothers.com states: “Vegan cheese is usually made from soy, nuts, vegetable oils, and various other natural ingredients like peas or arrowroot”.

It follows that a wide range of ingredients have been proposed for incorporation into cheeses. However, in general terms, vegan cheeses tend to be relatively low in protein and may include a wide range of chemical additives, to achieve desirable properties of, for example, texture, taste, and colour, amongst others.

Desirable milk-based cheeses include: semi-hard cheeses such as mozzarella, gouda and edam; hard cheeses, such as cheddar, emmental and gruyere; and extra hard cheeses such as parmesan. Such “hard” cheeses exhibit a range of desirable properties such as sliceability, grateability, excellent taste (when raw or cooked), and visual appeal (when raw or cooked). In addition, milk-based hard cheeses contain a high level of protein which is able to deliver a wide-range of amino acids.

Whilst it is desirable to produce hard (eg semi-hard through to extra hard) vegan cheeses having the properties described, it is very challenging. In particular, it is difficult to produce a vegan cheese which has high levels of protein and characteristics which largely correspond to those of milk-based cheeses. For example, it is very challenging to produce a vegan cheese which closely replicates a hard cheese in having appropriate hardness and chewiness; and, furthermore, exhibits those characteristics whilst at the same time exhibiting other desirable characteristics, such as being visually attractive and being sliceable and grateable. In addition, it is desirable that a hard, vegan cheese can be cooked so as to exhibit a desirable colour, such as a golden colour, as is generally found with milk-based hard cheeses. It is also desirable that the vegan cheese has an appropriate flavour, for example an appropriate cheese-like flavour, without the need to include high levels of flavourings or other taste-masking ingredients.

Another property which is desirable for cheese in general but which can be difficult to achieve in a hard, vegan cheese, is meltability, for example when heated in a conventional thermal oven or a microwave oven.

It is an object of preferred embodiments of the present invention to address the above-mentioned problems.

It is an object of preferred embodiments of the present invention, to provide a hard, vegan cheese.

It is an object of preferred embodiments of the present invention to provide a hard and/or chewy vegan cheese.

It is an object of preferred embodiments of the present invention to provide a hard and meltable vegan cheese.

It is an object of preferred embodiments of the present invention to provide a vegan cheese which is visually attractive and desirable in a chilled or melted state.

It is an object of preferred embodiments of the present invention to provide a vegan cheese which is processable, for example, grateable and sliceable at its point of use.

It is an object of preferred embodiments of the present invention to provide a vegan cheese which is not heavily starch-based or nut-based.

According to a first aspect of the invention, there is provided a foodstuff, for example a vegan cheese, the foodstuff comprising:
a fungus; and
an oil.

Said oil is suitably primarily composed of fatty acids in the form of esters. References below to an oil which is composed of or includes a fatty acid includes the fatty acid as an ester (e.g. the oil include a fatty acid residue), rather than the referenced fatty acid being a free fatty acid in the oil.

Said oil is preferably a vegetable oil.

Said oil may have a melting point of at least 20 °C, preferably at least 22 °C. The melting point may be less than 40 °C, preferably less than 30 °C, more preferably less than 27 °C.

Said oil has preferably not been chemically treated to functionalise it. It is preferably not a hydrogenated version of a natural oil.

The % of components in an oil as described herein may be assessed by GC-HRMS or alternative techniques known to a skilled person in the art.

Said oil may include at least 60 wt %, preferably at least 70 wt %, more preferably at least 78 wt %, of saturated fatty acids. Said oil may include less than 95 wt %, preferably less than 90 wt %, of saturated fatty acids.

Said oil may include at least 4 wt % of monounsaturated fatty acids. Said oil may include less than 10 wt %, preferably less than 8 wt %, of monounsaturated fatty acids.

Said oil may include less than 5 wt %, preferably less than 3 wt %, of polyunsaturated fatty acids.

Said oil may include at least 40 wt %, preferably at least 45 wt %, of lauric acid. Said oil may include less than 65 wt %, preferably less than 60 wt %, of lauric acid.

Said oil may include at least 15 wt % of myristic acid. Said oil may include less than 30 wt %, preferably less than 25 wt %, of myristic acid.

Said oil may include at least 6 wt % of palmitic acid. Said oil may include less than 15 wt %, preferably less than 11 wt %, of palmitic acid.

Said oil may include at least 4 wt % of oleic acid. Said oil may include less than 10 wt %, preferably less than 8 wt %, of oleic acid.

Said oil may include at least 2 wt %, preferably at least 3 wt, of stearic acid. Said oil may include less than 5 wt %, of stearic acid.

Said oil may include at least 4 wt % of capric acid. Said oil may include less than 10 wt %, preferably less than 8 wt %, of capric acid.

Said oil may include at least 1 wt %, preferably at least 2 wt %, of linoleic acid. Said oil may include less than 5 wt %, of linoleic acid.

Any grade of said oil, for example virgin, extra virgin, highly refined or winterized, may be selected.

Said oil is preferably coconut oil.

Said oil preferably makes up at least 80 wt %, preferably at least 90 wt %, more preferably at least 95 wt % or, especially, at least 99 wt % of the total wt % of all oils in said foodstuff, excluding any oil or fat in the fungus. Said oil may be the only oil in the foodstuff, excluding any oil or fat in the fungus.

Said foodstuff may include at least 15 wt %, preferably at least 19 wt %, more preferably at least 21 wt %, especially at least 23 wt % of said oil. Said foodstuff may include less than 40 wt %, preferably less than 35 wt %, more preferably less than 32 wt % of said oil.

The sum of the wt % of all oils and fats in said foodstuff excluding any oil/fat in the fungus may be at least 15 wt %, preferably at least 19 wt %, more preferably at least 21 wt %, especially at least 23 wt %. The sum of the wt % of all oils and fats in said foodstuff excluding any oil/fat in the fungus may be less than 40 wt %, preferably less than 35 wt %, more preferably less than 30 wt %.

Unless otherwise stated herein, the amount of fungus is on a dry mass basis. That is the weight on a dry mass basis excludes any water which may be incorporated into a mass comping the fungus.

Said fungus may include at least 35 wt %, preferably at least 40 wt %, protein on a dry mass basis. Said fungus may include less than 65 wt %, preferably less than 60 wt %, protein on a dry mass basis.

Said fungus may include at least 10 wt %, preferably at least 15 wt %, dietary fibre on a dry mass basis. Said fungus may include less than 30 wt %, preferably less than 23 wt %, dietary fibre on a dry mass basis.

Said fungus may include at least 4 wt %, preferably at least 7 wt %, fat on a dry mass basis. Said fungus may include less than 20 wt %, preferably less than 10 wt %, fat on a dry mass basis.

Preferably, said fungus is a filamentous fungus.

Said fungus may comprise filaments. Said filaments may have lengths of less than 500 μm, preferably less than 100 μm. Said filaments may have a length greater than 1 μm, for example greater than 10 μm. Preferably, fewer than 5 wt %, preferably substantially no, filaments of said filamentous fungus have lengths of greater than 500 μm; and preferably fewer than 5 wt %, preferably substantially no filaments have lengths of greater than 250 μm. Preferably, values for the number average of the lengths of said filaments are also as stated above. Thus, the number average of the lengths of said filaments may be in the range 1 μm to 100 μm, preferably in the range 2 μm to 80 μm.

Said filamentous fungus may comprise filaments having diameters of less than 20 μm, preferably less than 10 μm, more preferably 5 μm or less. Said filaments may have diameters greater than 1 μm, preferably greater than 2 μm. Preferably, values for the number average of said diameters of said filaments are also as stated above. Thus, the number average of said diameters of said filaments are preferably in the range 1 μm to 20 μm.

Said filamentous fungus may comprise filaments having an aspect ratio (length/diameter) of less than 500, preferably less than 200, more preferably less than 100. The aspect ratio may be greater than 5, preferably greater than 10. Preferably, values for the average aspect ratio of said filaments (i.e. the number average of the lengths of the filaments divided by the number average of the diameters of the said filaments) are also as stated above. Thus, the number average of the lengths of the filaments divided by the number average of the diameters of the said filaments is preferably in the range 5 to 500, more preferably in the range 10 to 100.

Preferably, said filamentous fungus comprises fungal mycelia and suitably at least 80 wt %, preferably at least 90 wt %, more preferably at least 95 wt % and, especially, at least 99 wt % of said filamentous fungus comprise fungal mycelia. Some filamentous fungi may include both fungal mycelia and fruiting bodies. Said filamentous fungus preferably comprise a filamentous fungus of a type which does not significant produce fruiting bodies.

Said filamentous fungus preferably comprises fungus selected from fungi imperfecti.

Preferably, said filamentous fungus comprises, and preferably consists essentially of, cells of Fusarium species, especially of Fusarium venenatum A3/5 (formerly classified as Fusarium graminearum) (IMI 145425; ATCC PTA-2684 deposited with the American Type Culture Collection, 10801 University Boulevard, Manassas, VA.).

Preferably, said filamentous fungus is non-viable. Preferably, said filamentous fungus has been treated to lower the level of RNA which it contains. Thus, the level of RNA in said filamentous fungus is preferably less than the level in an identical fungus when in a viable state. The level of RNA in the filamentous fungus is preferably less than 2 wt % on a dry mass basis.

Said foodstuff suitably includes at least 1.0 wt %, preferably at least 2.0, of said fungus on a dry mass basis. Said foodstuff may include less than 30 wt %, suitably less than 25 wt %, preferably less than 20 wt %, more preferably less than 17 wt %, of said fungus on a dry mass basis.

Said foodstuff suitably includes at least 1.0 wt %, preferably at least 2.0 wt %, of protein on a dry mass basis. Said foodstuff may include less than 30 wt %, suitably less than 25 wt %, preferably less than 20 wt %, more preferably less than 17 wt %, of protein on a dry mass basis. The protein level may be assessed by the Kjeldahl method.

Preferably, in said foodstuff, the sum of the wt % of said fungus on a dry mass basis and said oil is at least 30 wt %, preferably at least 35 wt %, more preferably at least 37 wt %. Said sum may be less than 50 wt %, preferably less than 45 wt %.

Preferably, in said foodstuff, the sum of the wt % of each fungus on a dry mass basis and each oil/fat is at least 30 wt %, preferably at least 35 wt %, more preferably at least 37 wt %. Said sum may be less than 50 wt %, preferably less than 45 wt %.

A reference herein to the weight of water in said composition suitably refers to the total water content in the composition irrespective of its origin.

Said foodstuff may include at least 5 wt %, preferably at least 10 wt % of water. In some cases, the foodstuff may include at least 15 wt % or at least 20 wt % of water. The amount of water in said foodstuff may be less than 50 wt % or less than 42 wt %.

In said foodstuff, suitably, a ratio defined as the weight of said oil divided by the weight of water is greater than 0.2, preferably is greater than 0.4. The ratio may be less than 3.0, preferably less than 2.5, more preferably less than 2.0.

In said foodstuff, suitably, a ratio defined as the total weight of oil(s)/fats(s) divided by the weight of water is greater than 0.2, preferably is greater than 0.4. The ratio may be less than 3.0, preferably less than 2.5, more preferably less than 2.0.

In said foodstuff, the sum of the wt % of said fungus, oil and water may be at least 50 wt %, preferably at least 60 wt %, more preferably at least 70 wt %. Said sum may be less than 95 wt %, preferably less than 90 wt %.

In said foodstuff, the sum of the wt % of each fungus, each oil/fat and water may be at least 50 wt %, preferably at least 60 wt %, more preferably at least 70 wt %. Said sum may be less than 95 wt %, preferably less than 90 wt %.

Said foodstuff may include:
1 to 30 wt %, preferably 2 to 20 wt %, of said fungus on a dry mass basis; and
15 to 40 wt %, preferably 21 to 31 wt %, of an oil.

The balance may include other ingredients including water and optionally one or more starches.

Said foodstuff suitably includes one or more starches. The sum of the wt % of starches in said foodstuff may be at least 4.0 wt %, preferably at least 8.0 wt %. Said sum may be less than 30.0 wt %, preferably less than 28.0 wt %. In one example, the sum of the wt % of starches in said foodstuff may be less than 20 wt % or less than 15 wt %, for example in the range 5 to 15 wt %.

The level of amylose in the one or more starches may affect the hardness of a foodstuff described. For example, starches which have higher levels of amylose (eg greater than 10.0 wt %, greater than 15.0 wt % or greater than 20.0 wt %) may facilitate production of harder foodstuffs, for example hard or extra hard foodstuffs. Examples of starches which include high levels of amylose include corn, potato and wheat starches. Of these, corn starch is most preferred. Starches which include lower levels of amylose (eg less than 10.0 wt %) may include waxy corn, rice or tapioca starches.

To produce a hard cheese, said foodstuff may include at least 1.0 wt % or at least 2.0 wt % (and suitably less than 15.0 wt % or less than 10.0 wt %) of a starch which has a relatively high level (eg greater than 20.0 wt %) of amylose as discussed. For semi-hard cheeses, the total wt % of starch(es) which include(s) lower levels of amylose (eg less than 10 wt %) (and higher levels of amylopectin) may be greater than the total wt % of starches in the foodstuff which include a high level of amylose (eg greater than 20.0 wt %).

Said foodstuff may be a hard, vegan cheese; or said foodstuff may be a semi-hard vegan cheese.

In one preferred embodiment, said foodstuff may include a starch which is relatively high in amylose. Said foodstuff may include a starch which has at least 20% wt % amylose. Such a starch may include less than 35 wt % or less than 30 wt % amylose.

Said starch is preferably vegetable-derived.

Said foodstuff may include at least one starch selected from corn starch, rice starch, waxy corn starch, tapioca starch, potato starch and wheat starch.

In said foodstuff, the sum of the wt % of each fungus, each oil/fat, each starch and water may be at least 70.0 wt %, preferably at least 80.0 wt %, more preferably at least 90.0 wt %, especially at least 93.0 wt %. Said sum may be less than 100 wt %, preferably less than 99.0 wt %, more preferably less than 98.0 wt %.

Said foodstuff may include one or more flavourants and/or colourants. For example, the sum of the wt % of flavourants and colourants in the foodstuff may be at least 1.0 wt %, preferably at least 2.0 wt %. Said sum may be less than 6.0 wt %.

Said foodstuff suitably has a carbohydrate content of less than 25 g/100 g or less than 20 g/100 g or less than 18 g/100 g.

Said foodstuff may include 0 to 5 wt % of edible hydrocolloid. Suitably, however, said foodstuff includes less than 4.0 wt %, preferably less than 2.0 wt %, more preferably less than 1.0 wt % and, especially, less than 0.5 wt % of hydrocolloids in total. It may include less than 0.1 wt % or even about 0 wt % of hydrocolloids in total.

Said foodstuff may include less than 4.0 wt %, preferably less than 2.0 wt %, more preferably less than 1.0 wt % and, especially, less than 0.5 wt % of non-starch based carbohydrate polymers in total. It may even include less than 0.1 wt % or even about 0 wt % of non-starch based carbohydrate polymers in total.

Said foodstuff may include less than 4.0 wt %, preferably less than 2.0 wt %, more preferably less than 1.0 wt % and, especially, less than 0.5 wt % of sucrose. It may include less than 0.1 wt % or even about 0 wt % of sucrose.

In some cases, an acid, for example a food grade acid, such as lactic acid or propionic acid (lactic acid being preferred) may be added to the foodstuff to reduce the pH of the foodstuff to less than 4.5, Said acid may increase shelf-life of the foodstuff and/or also act to soften the texture of the foodstuff. In a preferred embodiment, lactic acid is added to reduce the pH of the foodstuff.

Said foodstuff may include less than 4.0 wt %, preferably less than 2.0 wt %, more preferably less than 1.0 wt % and, especially, less than 0.5 wt % of lactic acid. It may include less than 0.1 wt % or even about 0 wt % of lactic acid.

Said foodstuff may include less than 4.0 wt %, preferably less than 2.0 wt %, more preferably less than 1.0 wt % and, especially, less than 0.5 wt % of lactic acid producing bacteria. It may include less than 0.1 wt % or even about 0 wt % of lactic acid producing bacteria.

Said foodstuff may include less than 4.0 wt %, preferably less than 2.0 wt %, more preferably less than 1.0 wt % and, especially, less than 0.5 wt % of bacteria. It may include less than 0.1 wt %, less than 0.01 wt % or even about 0 wt % of bacteria.

Said foodstuff may include one or more mineral additives (in addition to any mineral additive which includes a Group I metal), for example, calcium, magnesium or ferrous salts. Said foodstuff preferably includes less than 2.0 wt %, preferably less than 1.0 wt %, more preferably less than 0.1 wt % and, especially, less than 0.05 wt % of said one or more mineral additives. It may include less than 0.01 wt % or even about 0 wt % of said one or more mineral additives.

Said foodstuff suitably has a protein content of at least 5 g/100 g, preferably at least 8 g/100 g, more preferably at least 10 g/100 g of protein. The protein content may be less than 25 g/100 g.

Said foodstuff preferably includes 0 wt % of ingredients of animal origin. Said foodstuff is preferably suitable for vegans. Said foodstuff is preferably a cheese-substitute which includes no animal products.

In said foodstuff, the sum of the wt % of each fungus, each oil/fat, each starch, each flavourant, salt (eg NaCl) and water may be at least 80.0 wt %, preferably at least 85.0 wt %, more preferably at least 90.0 wt %, especially at least 95 wt %. Said sum may be 100 wt % or less.

Said foodstuff may be in the form of a block. The block may have a volume of at least 25 cm³, preferably at least 100 cm³. The volume may be less than 10000 cm³, less than 1000 cm³ or less than 500 cm³. The block may have at least one opposing pair of substantially planar faces.

In some cases, said block may have a weight of up to 650 Kg. The weight may be in the range 100 g to 650 Kg, for example in the range 200 g to 20 Kg.

Said foodstuff, for example said block, may have a hardness, assess as described in Test 1, of at least 1000 g, at least 2000 g or at least 3000 g. The hardness may be less than 40000 g, less than 35000 g or less than 30000 g. A semi-hard cheese may have a hardness in the range 1000 g to 12000 g. A hard cheese may have a hardness of more than 12000 g. A hard cheese may have a hardness in the range 12000 to 40000 g or in the range 12000 to 30000 g. Hardness is suitably assessed at 20 °C.

Said foodstuff is preferably provided in a package. For example, a said block may be provided in said package. The package may be arranged to restrict passage of oxygen to the foodstuff therein. Said package may comprise an oxygen barrier material. Said package may comprise a plastics packaging material. Said foodstuff is preferably the only foodstuff contained in the package.

According to a second aspect of the invention, there is provided a process for making a foodstuff, the process comprising mixing a fungus and an oil.

Said foodstuff may have any feature of the foodstuff of the first aspect. Said fungus may have any feature of the fungus of the first aspect. Said oil may have any feature of the oil of the first aspect.

In a step (i), a biomass comprising said fungus is suitably selected. Said biomass may be in the form of a paste. Said biomass may include 20-30 wt %, (preferably 22-26 wt %) of fungus on a dry mass basis and 70-80 wt %, (preferably 74 to 78 wt %) of water.

Said process may include contacting the fungus with said oil, suitably with mixing.

Said process may include contacting the fungus with a starch as described in the first aspect.

Said process may include contacting the fungus with one or more flavourants and/or colourants as described according to the first aspect.

Said process may comprise introducing a mixture prepare in the process into a mould, for example to form said mixture into a predetermined shape, for example into a block.

Said process is preferably for producing a foodstuff having a hardness, suitably assessed as described in Test 1, as described in the first aspect.

Said process is preferably for producing a foodstuff which is a vegan cheese and is suitably processable, for example, grateable and sliceable at its point of use.

Any feature of any aspect of any invention described herein may be combined with any feature of any other invention described herein mutatis mutandis.

Specific embodiments of the invention will now be described, by way of example, with reference to the accompanying figures in which:

FIG. 1 is a graph comparing hardnesses of the cheeses of Examples 1 to 6;

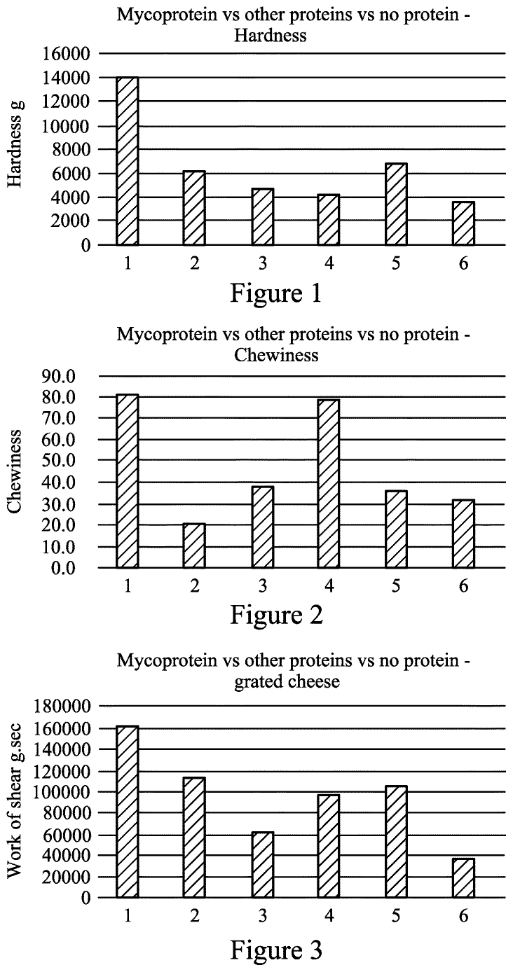

FIG. 2 is a graph comparing the chewiness of the cheeses of Examples 1 to 6;

FIG. 3 is a graph comparing Work of shearing for cheeses of Examples 1 to 6;

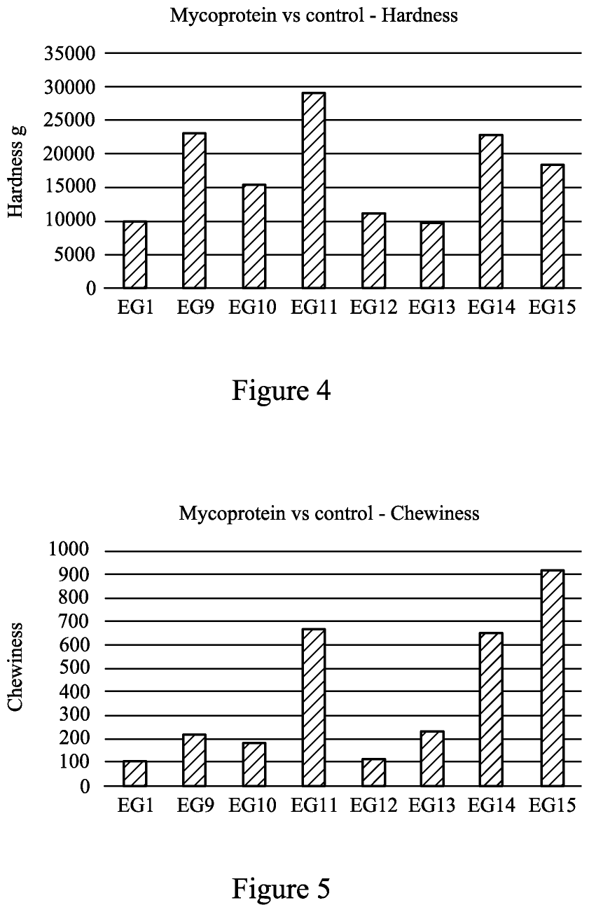

FIG. 4 is a chart comparing Hardnesses for vegan cheeses as described in preferred embodiments and commercially-available cheeses;

FIG. 5 is a chart comparing Chewiness for vegan cheeses as described in preferred embodiments and commercially-available cheeses;

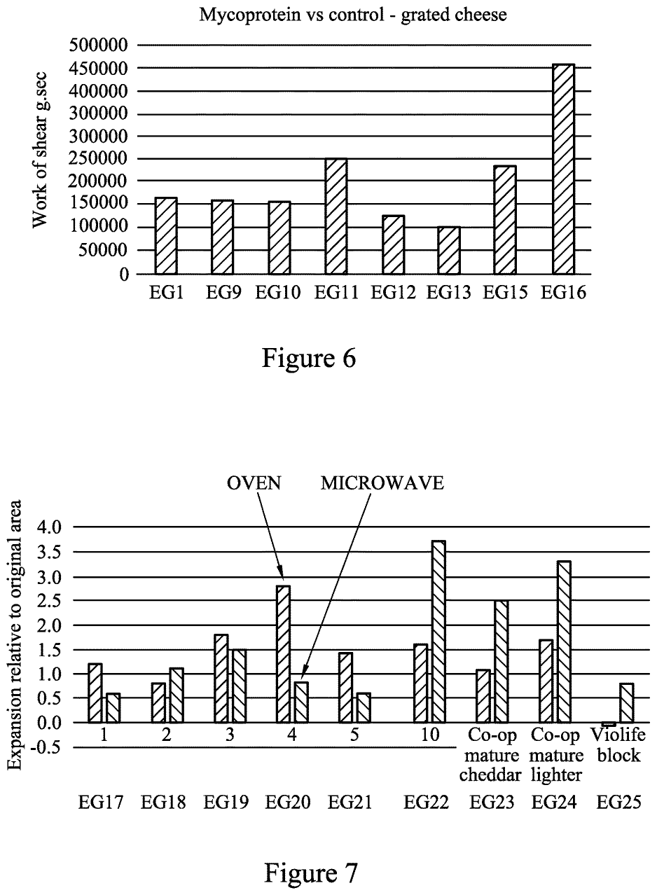

FIG. 6 is a chart comparing Work of Shear for vegan cheeses as described in preferred embodiments and commercially-available cheeses; and

FIG. 7 is a chart comparing meltability for vegan cheeses as described in preferred embodiments and commercially-available cheeses.

The following materials are referred to herein:

Mycoprotein paste-Mycoprotein paste-refers to a visco-elastic material comprising a mass of edible filamentous fungus derived from Fusarium venenatum A3/5 (formerly classified as Fusarium graminearum Schwabe) (IMI 145425; ATCC PTA-2684 deposited with the American type Culture Collection, 12301 Parklawn Drive, Rockville Md. 20852) and treated to reduce its RNA content to less than 2% by weight by heat treatment. Further details on the material are provided in WO96/21362 and WO95/23843. The material may be obtained from Marlow Foods Limited of Stokesley, U.K. It comprises about 23-25 wt % solids (the balance being water) made up of non-viable RNA reduced fungal hyphae of approximately 400-750 μm length, 3-5 μm in diameter and a branching frequency of 2-3 tips per hyphal length.

Coconut oil—Kirkland™ Coconut Oil Unrefined Virgin Cold Pressed 100% Pure, having a melting point of about 24 °C. An alternative is CocoPacific™ Odourless Raw Extra Virgin Coconut Oil.

Corn starch—a corn starch with an amylose content of about 25 wt %.

Rice starch—a waxy rice starch with a negligible amylose content.

Potato protein—a potato protein with a total protein content of 90.5 g/100 g.

Nutritional yeast—a commercially-available yeast.

Pea protein—Pisane™ M9, from Fibrisol™, having a protein content of 81.7 g/100 g.

Faba protein—product from Australian Plant Protein, having a protein content of 82.1 g/100 g.

Soy protein—product having a protein content of 90.0 g/100 g.

The following tests and assessments are referred to herein.

### Test 1—Texture Profile Analysis (TPA)

TPA was used as it is found to provide a good comparison to actual sensory characteristics,

Products were cut into 1 inch (2.5 cm) discs to define samples for testing. The samples were of uniform thickness of 11 mm. Samples were stored in an 8 °C chiller, and tested within 5 minutes of removal.

TPA was performed using a TA. XT Plus Texture Analyser (Stable Micro Systems, Godalming UK) and a stainless-steel compression platen of 75 mm diameter (Stable Micro Systems, Godalming UK). The platen attachment was used to compress each sample using the standard ‘Simplified TPA’ method, found within the Exponent software from Stable Micro Systems; a modified version of the original instrumental test method created by A. Szczesniak (1963) and General Foods Corporation Technical Centre in 1963—see SZCZESNIAK, A. S., BRANDT, M. A. and FRIEDMAN, H. H. (1963), Development of Standard Rating Scales for Mechanical Parameters of Texture and Correlation Between the Objective and the Sensory Methods of Texture Evaluation. Journal of Food Science, 28:397-403. A TPA double compression test was performed with compression more than half of the sample thickness and the sample was compressed twice to allow observation on springiness or bounciness upon compaction from the first compression. The test was undertaken at ambient temperature (eg 20 °C.+/−5 °C).

Parameters used for the method were as detailed in the table below.

| Pre-Test Speed | 1.00 | mm/sec |
|---|---|---|
| Test Speed | 5.00 | mm/sec |
| Post-Test Speed | 5.00 | mm/sec |
| Compression Percentage | 70% | |
| Time Between Compressions | 5.00 | seconds |
| Trigger Force | 5 | g |

Samples were compressed using the compression platen to a percentage of 70% at a speed of 5.00 mm/sec, using a 2-cycle analysis which allowed a 5 second gap between compressions. The deformation curve of each sample was obtained, and results used to determine the mechanical parameters of the samples, including; Hardness, Resilience, Cohesiveness, Springiness and Chewiness. The five characteristics were calculated by the ‘Simplified TPA’ macro included in the Exponent software from Stable Micro Systems. Each textural/mechanical parameter is explained in the table below, with reference to Texturetechnologies.com. (2019). Texture Profile Analysis. [online] Available at: https://texturetechnologies.com/resources/texture-profile-analysis #select-characteristics [Accessed 5 Nov. 2019].

| | |
|---|---|
| Hardness | Peak Force 1 |
| Resilience | Area 4/Area 3 |
| Cohesiveness | Area 2/Area 1 |
| Springiness | Distance 2/Distance 1 |
| Chewiness | Hardness × Cohesiveness × Springiness |

### Test 2—Methodology for Kramer Shear Cell Testing

Kramer shear cell testing was used to validate results of Test 1.

Products were cut into 20 mm×20 mm×50 mm rectangles to define samples for testing-Samples were stored in a 5 °C chiller, and brought to room temperature before testing (18 °C).

The Kramer shear cell method was performed using a TA.HD Texture Analyser (Stable Micro Systems, Godalming UK) and a 5 bladed probe with a Kramer cell.—The blade attachment was used to compress each sample using the standard “Typical Kramer shear cell test” method, found within the Exponent software from Stable Micro Systems.

Parameters used for the method were as detailed in the table below.

| | | |
|---|---|---|
| Pre-Test Speed | 2.00 | mm/sec |
| Test Speed | 2.00 | mm/sec |
| Distance | 25 | mm |
| Trigger Force | 30 | g |

Samples were compressed using the 5 blade attachment to a depth of 25 mm at a speed of 2.00 mm/sec. The deformation curve of each sample was obtained, and results used to determine the mechanical parameters of the samples, including, Maximum shear force and work of shearing.

### Test 3—Sensory Analysis

A panel was assembled in accordance with ISO 8586: 2012 (E) (Corrected version 2014 Jun. 15) and trained to undertake a range of sensory tests on selected foodstuffs. In particular, a blind tasting of samples was undertaken.

### Test 4—Sliceability

Samples were sliced with a knife to 3 mm and packing on the knife and crumbling of the cheese were assessed.

Test 5—Colour on cooking

Slices were heated in a frying pan on medium heat to test for colour on cooking and in particular browning.

### Test 6—Grateability

A 100 g block was grated in a MagiMix (large grate size) and tested in a Kramer shear cell (5-bladed probe) with the following parameters.

| | | |
|---|---|---|
| Pre-Test Speed | 2.00 | mm/sec |
| Test Speed | 3.00 | mm/sec |
| Post-Test Speed | 10.00 | mm/sec |
| Distance | 70 | mm |
| Trigger Force | 10 | g |

Test 7—Assessment of meltability of cheeses

In general terms, meltability of samples was assessed by comparing the area of a sample before and after heating. In the method, samples were cut from a mass into discs of 25 mm diameter and 11 mm height. The discs were placed within suitable trays and heated either in a conventional thermal oven at 180 °C for five minutes or in a microwave oven at 800 W for 10 seconds if an individual disc was assessed; or at 800 W for 20 seconds if four discs were assessed together. Images of the discs before and after heating were captured using a iPhone 11 camera and images exported to commercially available Image J™ software. All images include an image of a ruler alongside the discs which allows the software to set the scale and calibrate the pixel ratio to unit length. The meltability was based on the expansion of the discs, calculated by:

$$\vert \frac{{A}_{unheated} - {A}_{heated}}{{A}_{unheated}} \vert$$

where A is the area.

### Test 8—Assessment of Total Water Content of Cheeses

The moisture % of cheeses was assessed by weighing 3 g of a sample and then re-weighing during removal of moisture by heating to 130 °C until no more weight was lost. The difference between the initial weight and final weight was used to calculate moisture content.

### Example 1—Preparation of Hard, Vegan Cheese

A vegan cheese was made from the ingredients in the table below.

| Ingredient | wt % |
|---|---|
| Mycoprotein paste | 63 |
| Coconut oil | 25 |
| Salt | 2 |
| Corn starch | 10 |

In the preparation, the mycoprotein paste and coconut oil were mixed at high speed in a Thermomix (Trade Mark) mixer at 37 °C for a minimum of 5 minutes until a homogenous, smooth mix is formed. Then, the corn starch and salt were added to the mixer and mixing continued at low speed for a further minute. Then the mixture was heated to 85 °C at mid to high speed to gelatinize the starch and yield a smooth, homogeneous mix. It could take between 5 and 10 minutes for the sample to reach temperature. The mixture was then poured into a mould in the form of a foil tray and chilled in a standard fridge at a temperature of 5 °C.

The cheese prepared had a protein content of 8.5 wt % and a water content, determined by use of a moisture analyser of 25.7%.

### Examples 2 to 6—Preparation of Alternate Cheeses Using Other Common Protein-Containing Ingredients or No Protein-Containing Ingredient (Comparative Examples)

Cheeses were made using other non-animal based protein-containing ingredients (or no protein-containing ingredients) and compared to the vegan cheese of Example 1.

Cheeses were prepared having the ingredients in the table below.

Example No.
wt %

| Ingredient | 2 | 3 | 4 | 5 | 6 |
|---|---|---|---|---|---|
| Water | 53.6 | 52.6 | 52.7 | 53.6 | 63 |
| Coconut oil | 25 | 25 | 25 | 25 | 25 |
| Salt | 2 | 2 | 2 | 2 | 2 |
| Corn starch | 10 | 10 | 10 | 10 | 10 |
| Potato | 9.4 | — | — | — | — |
| protein | | | | | |
| Pea protein | — | 10.4 | — | — | — |
| Faba protein | — | — | 10.4 | — | — |
| Soy protein | — | — | — | 9.5 | — |

In the preparation, the water and coconut oil were mixed at high speed in a Thermomix (Trade Mark) mixer at 37 °C for a minimum of 5 minutes until a homogenous, smooth mix was formed. Then, the selected protein, starch and salt were added to the mixer and mixing continued at low speed for a further minute. Then the mixture was heated to 85 °C at mid to high speed to gelatinize the starch and yield a smooth, homogeneous mix. It could take between 5 and 10 minutes for the sample to reach temperature. The mixture was then poured into a mould in the form of a foil tray and chilled in a standard fridge at a temperature of 5 °C.

The table below includes information on the levels of specified components and characteristics of the vegan cheeses of Examples 1 to 6. Nutritional information was calculated from the raw ingredients' nutrient data having regard to the wt % included in the cheeses.

| | Example No. | | | | | |
|---|---|---|---|---|---|---|
| Characteristic | 1 | 2 | 3 | 4 | 5 | 6 |
| Energy (kcal) | 308.6 | 291.5 | 299.5 | 298.5 | 293.4 | 256.5 |
| Protein g/100 g | 8.2 | 8.5 | 8.5 | 8.5 | 8.5 | 0 |
| Fat -Total | 26.0 | 25.0 | 25.9 | 25.6 | 25.2 | 25.0 |
| g/100 g | | | | | | |
| Carbohydrate - | 9.7 | 8.8 | 8.9 | 9.2 | 8.9 | 8.8 |
| Total g/100 g | | | | | | |
| Fibre -Total | 3.7 | 0 | 0.2 | 0 | 0.2 | 0 |
| g/100 g | | | | | | |

### Example 7—Testing of Cheeses of Examples 1 to 6

A texture profile analysis of each cheese of Examples 1 to 6 was undertaken as described in Test 1 and results for the hardness and chewiness are provided in FIGS. 1 and 2.

FIG. 1, for Example 6 which contains no protein, shows the lowest value, suggesting that protein is playing a role in the firmness of the cheeses. Example 1, incorporating mycoprotein, has a significantly greater hardness than Example 2 to 5, which utilize different plant-based proteins to match the protein content of Example 1. This implies that mycoprotein has a technological advantage in creating a firm cheese over other plant proteins.

FIG. 2 illustrates chewiness, which is key in many firm cheeses, and often lacking in prior art vegan cheeses. Again, the mycoprotein-containing Example 1 shows the greatest chew in comparison to the others, which is a technological advantage.

Grateability of each cheese of Examples 1 to 6 was undertaken as described in Test 6 and results are provided in FIG. 3. The FIG. 3 illustrates the durability of the grated cheese samples. Example 6 has an extremely low work of shear, as the grated cheese had very little structure, whereas the mycoprotein-containing Example 1 has the greatest work of shear. This may be advantageous, for example, in a bag of pre-grated cheese, where the grated strands must maintain integrity.

### Example 8—Other Tests Undertaken on Cheeses of Examples 1 to 6

Tests 3 to 6 were undertaken on samples of the cheeses of Examples 1 to 6 and observations are summarised in the table below.

| | Example number | | | | | |
|---|---|---|---|---|---|---|
| Test | 1 | 2 | 3 | 4 | 5 | 6 |
| Visual | Brownish - | Dark brown | Good colour - | Greenish- | Good colour - | White, gel- |
| | can see | colour and | includes holes | can see | can see | like (starchy) |
| | crystals of fat | appears grainy | | crystals of | crystals of | |
| | | | | fat | fat | |
| Grating | Grated best | Grated best | Inferior | Inferior | Inferior | Inferior |
| | | | gratability - | gratability | gratability | gratability, |
| | | | soft, packing | soft, packing | soft, packing | very soft, lots |
| | | | on grater | on grater | on grater | of packing |
| Slicing | Sliced well | Sliced well | Sliced well, | Sliced well, | Sliced well, | Sliced |
| | | | some | some | some | acceptably, |
| | | | packing on | packing on | packing on | but packing |
| | | | knife | knife | knife | on knife |
| Frying | Turns a | Slightly | Slightly | Slightly | Turns a | Large starch |
| | golden | darkened | darkened | darkened | golden | bubbles, no |
| | colour on | on frying | on frying | on frying | colour on | browning |
| | frying | | | | frying | |
| Taste | Generally | Grainy, | Powdery, | Powdery, | Less powdery | Very |
| (raw) | neutral taste. | powdery, | bean off- | bean off- | than Examples | starchy/gel- |
| | Lactic notes | overwhelming | notes; soft; | notes; soft | 2 to 4; and | like |
| | which give the | off-notes | not a cheesey | | more neutral | |
| | impression of | | eat | | flavour; but | |
| | cheese; a | | | | bland, | |
| | little savoury | | | | acceptable | |
| | flavour | | | | mouthfeel | |
| Taste | Very cheesy | As for raw; but | Dried all the way | Some | More powdery | Very |
| (fried) | (flavour and | biscuity/crunchy | through; eats like | crispiness, | following | starchy/gel- |
| | texture); good | on fried side | a biscuit/cracker; | still powdery | frying; good | like |
| | golden crisp | | flavour neutralised | as for raw | crispiness | |
| | on fried side | | a little | with pea off- | and colour | |
| | | | compared to raw | notes | | |

### Examples 9 to 13-Preparation of Other Mycoprotein-Containing Hard or Semi-Hard Vegan Cheeses

Following the procedure generally described for Example 1, vegan cheeses of Examples 9 to 11 were prepared having the ingredients detailed in the table below.

Examples 12 and 13, detailed in the table below, used a microfluidised mycoprotein paste instead of the mycoprotein paste. The preparation of the microfluidised paste involved homogenising chilled, fresh mycoprotein paste with cold tap water in a Thermomix™ mixer (ratio of 1 part paste to 2 parts water). This mixture was then sent through a Microfluidics™ microfluidizer at approximately 900 bar. The resultant milk (comprising 7.77 wt % solids) was then homogenised in the Thermomix mixer again, and additional paste was added to adjust the ratio to 1 part paste to 1 part water, the mixture then comprising 11.14 wt % solids.

Next in the preparation, the microfluidised paste and coconut oil were mixed at high speed in a Thermomix (Trade Mark) mixer at 37 °C for a minimum of 5 minutes until a homogenous, smooth mix was formed. Dry ingredients were then added to the mixer and mixing continued at low speed for a further minute. Then the mixture was heated to 85 °C at mid to high speed to gelatinize the starch and yield a smooth, homogeneous mix. It could take between 5 and 10 minutes for the sample to reach temperature. The mixture was then poured into a mould in the form of a foil or silicone tray and chilled in a standard fridge at a temperature of 5 °C.

Example No.
wt %

| Ingredient | 9 | 10 | 11 | 12 | 13 |
|---|---|---|---|---|---|
| Mycoprotein | 54 | 58 | 48 | 27 | 29.5 |
| paste | | | | | |
| Water | 0 | 0 | 0 | 27 | 29.5 |
| Coconut oil | 30 | 30 | 30 | 30 | 25 |
| Salt | 1 | 2 | 2 | 1 | 1 |
| Corn starch | 10 | 10 | 20 | 10 | 10 |
| Nutritional yeast | 5 | 0 | 0 | 5 | 5 |

The table below includes information on the levels of specified components and characteristics of the vegan cheeses of Examples 9 to 13, with relevant characteristics being assessed as described in Tests 7 to 13 as applicable. In addition, the table details the levels of specified components and characteristics of commercially-available vegan cheeses (VIOLIFE™ block-Example 14; and VIOLIFE™ grated-Example 16) and a dairy cheese (Co-op™ cheddar-Example 15).

| | Example No. | | | | | | | |
|---|---|---|---|---|---|---|---|---|
| Characteristic | 9 | 10 | 11 | 12 | 13 | 14 | 15 | 16 |
| Energy (kcal) | 363.3 | 349.2 | 373.9 | 341.0 | 298.4 | 328.0 | 416.0 | 298.0 |
| Protein g/100 g | 9.4 | 7.5 | 6.2 | 5.9 | 6.2 | 0.4 | 25.0 | 0 |
| Fat -Total | 31.1 | 30.9 | 30.7 | 30.7 | 25.7 | 26.0 | 35.0 | 24.0 |
| g/100 g | | | | | | | | |
| Carbohydrate - | 10.6 | 9.7 | 18.3 | 10.2 | 10.2 | 23.0 | 0.5 | 21.0 |
| Total g/100 g | | | | | | | | |
| Fibre -Total | 4.2 | 3.4 | 2.8 | 2.6 | 2.7 | 0 | 0 | 0 |
| g/100 g | | | | | | | | |
| Solids wt % | 58.13 | 52.37 | 62.84 | 53.6 | 47.75 | 51.23 | 61.54 | — |

A texture profile analysis of each cheese of Examples 9 to 16 was undertaken as described in Test 1 and results for the hardness and chewiness are provided in FIGS. 4 and 5. FIG. 4 appears to show that:
(a) It is possible to achieve the hardness found in dairy cheddar and plant-based cheese using mycoprotein and it is possible to adjust hardness dependent on which cheese it is desired to mimic.
(b) Solid fat content may increase firmness (compare Examples 1 with Example 10 and Example 12 with Example 13).
(c) Increasing starch inclusion % may increase firmness (compare Examples 10 and 11).
(d) The total solids % relates to firmness (compare Examples 11 and 12).

FIG. 5 appears to show that:
(a) It is possible to match/beat the chewiness of Violife™ block whilst delivering a far higher level of protein.
(b) Corn starch may play a role in chewiness within the matrix.

Grateability of each cheese of Examples 9 to 16 was undertaken as described in Test 6 and results are provided in FIG. 6.

### Examples 17 to 22—Preparation of Hard or Semi-Hard, Meltable Vegan Cheeses

Vegan cheeses were made from the ingredients in the table below using the method as described for Examples 12 and 13.

| | Example No. | | | | | |
|---|---|---|---|---|---|---|
| | wt % | | | | | |
| Ingredient | 17 | 18 | 19 | 20 | 21 | 22 |
| Mycoprotein | 17.5 | 17.5 | 17.5 | 17.5 | 17.5 | 17.0 |
| paste | | | | | | |
| Water | 31.7 | 31.5 | 36.5 | 35.3 | 34.4 | 35.0 |
| Coconut oil | 25.0 | 25.0 | 25.0 | 25.0 | 25.0 | 25.0 |
| Salt | 1.0 | 1.0 | 1.0 | 1.0 | 1.0 | 1.5 |
| Corn starch | 2.2 | 5.0 | 0 | 0 | 4.9 | 1.5 |
| Rice starch | 19.8 | 20.0 | 20.0 | 16.2 | 14.5 | 20.0 |
| Nutritional yeast | 2.8 | 0 | 0 | 5.0 | 2.8 | 0 |

The table below includes information on the levels of specified components and characteristics of the vegan cheeses of Examples 17 to 22, with relevant characteristics being assessed as described in Tests 7 to 13 as applicable.

| | Example No. | | | | | |
|---|---|---|---|---|---|---|
| Characteristic | 17 | 18 | 19 | 20 | 21 | 22 |
| Energy (kcal) | 325.1 | 325.3 | 308.8 | 312.8 | 315.0 | 313.3 |
| Protein g/100 g | 3.7 | 2.4 | 2.4 | 4.7 | 3.6 | 2.3 |
| Fat -Total | 25.4 | 25.3 | 25.3 | 25.5 | 25.4 | 25.3 |
| g/100 g | | | | | | |
| Carbohydrate - | 20.2 | 22.3 | 17.9 | 15.5 | 17.8 | 19.2 |
| Total g/100 g | | | | | | |
| Fibre -Total | 1.6 | 1.0 | 1.0 | 2.0 | 1.6 | 1.0 |
| g/100 g | | | | | | |

### Examples 23 to 25—Comparative Commercially Available Cheeses

The following commercially available cheeses were assessed and the table below includes nutritional and other information.

Example No.

| Characteristic | 23 | 24 | 25 |
|---|---|---|---|
| Identity of | Co-op ™ | Co-op ™ | VIOLIFE ™ |
| cheese | MATURE | MATURE | BLOCK |
| | CHEDDAR | LIGHTER | |
| | | CHEDDAR | |
| Energy (kcal) | 416.0 | 319.0 | 328.0 |
| Protein g/100 g | 25.0 | 29.0 | 0.4 |
| Fat -Total | 35.0 | 22.0 | 26.0 |
| g/100 g | | | |
| Carbohydrate - | 0.5 | 0.5 | 23.0 |
| Total g/100 g | | | |
| Fibre -Total | 0 | 0 | 0 |
| g/100 g | | | |

### Example 26—Testing of Cheeses of Examples 17 to 22 and Comparison with Commercially Available Cheeses of Examples 23 to 25

The cheeses of example 17 to 22 and the commercially available cheeses of Examples 23 to 25 were subjected to Test 14 to assess meltability. Results are provided in FIG. 7 which shows the cheeses of examples 17 to 22 have meltability both when using a thermal oven or a microwave oven comparable to the conventional dairy-based Cheddar cheeses (Examples 23 and 24) whereas the meltability for the vegan cheese (Example 25) was relatively poor.

In addition, other properties of the cheeses of example 17 to 22 were assessed and were found to be comparable to dairy-based hard cheeses. In particular, it was found that the vegan cheeses of examples 17, 18, 21 and 22 were hard cheeses and the vegan cheeses of examples 19 and 20 were semi-hard cheeses.

### Example 27—Preparation of Meltable Vegan Cheese

Following the procedure generally described for Example 1, A vegan cheese having the following composition was prepared:

| Ingredient | Wt % |
|---|---|
| Mycoprotein | 28.35 |
| paste | |
| Water | 28.35 |
| Coconut oil | 23.5 |
| Salt | 1.5 |
| Corn starch | 1.5 |
| Rice starch | 16.5 |
| Sugar | 0.3 |

It was found that inclusion of the sugar improved the browning of the foodstuff.

The inventors have observed and/or speculatively conclude as follows:
Fat
   Increasing levels of solid fat may increase firmness/hardness.
   Increase in fat content may increase meltability in both a microwave and thermal oven.
Water content
   Increase in water content may reduce firmness/hardness and resilience.
   Increase in water content may increase spread in oven and microwave
Mycoprotein paste
   Reduction in hyphae length may yield a smoother vegan cheese texture, allow better incorporation of fat, prevent oiling off, and may affect meltability.
   Increasing the amount of mycoprotein paste may increase resilience and chewiness.
   Increasing the amount of mycoprotein paste may reduce meltability and stretchability.
Starch
   Addition of starch increases firmness, with higher amylose starches having a greater effect on firmness.
   Addition of starch increases resilience and chew, with higher amylose starches increasing both to a greater degree.
   Increasing levels of starch reduces meltability, with higher amylose starches inhibiting meltability to a greater degree than low amylose starches.
   Low amylose starches tend to increase stretchability.

### Meltability

Typically for a meltable, hard cheese, the total amylose content in a vegan cheese may be above 1 wt %, whilst in a semi-hard cheese it may be in the range 0.5-1 wt %. For a generally non-melting vegan cheese, the total amylose content may be in the range 2.5-5 wt %.

In general terms, the results show that vegan cheeses as described herein containing mycoprotein paste can be matched with commercially available milk-based cheddar cheeses. The mycoprotein-based vegan cheeses advantageously deliver high levels of protein and have excellent taste, texture, hardness, chewiness and aesthetic appeal in raw and cooked states.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A foodstuff, for example a vegan cheese, the foodstuff comprising:
a fungus; and
an oil.

2. A foodstuff according to claim 1, wherein said oil is a vegetable oil and has a melting point of at least 20 °C and a melting point of less than 40 °C, and/or
said oil includes at least 60 wt % of saturated fatty acids and less than 95 wt % of saturated fatty acids; and/or
said oil includes at least 4 wt % of monounsaturated fatty acids and less than 10 wt % of monounsaturated fatty acids; and/or
said oil includes less than 5 wt % of polyunsaturated fatty acids.

3. (canceled)

4. A foodstuff according to claim 2, wherein said oil includes:
at least 40 wt % of lauric acid and less than 65 wt % of lauric acid; and/or
at least 15 wt % of myristic acid and less than 30 wt % of myristic acid; and/or
at least 6 wt % of palmitic acid and less than 15 wt % of palmitic acid; and/or
at least 4 wt % of oleic acid and less than 10 wt % of oleic acid; and/or
at least 2 wt % of stearic acid and less than 5 wt % of stearic acid; and/or
at least 4 wt % of capric acid and less than 10 wt % of capric acid; and/or
at least 1 wt % of linoleic acid and less than 5 wt % of linoleic acid.

5. A foodstuff according to claim 1, wherein said oil is coconut oil.

6. A foodstuff according to claim 1, wherein:
said foodstuff includes at least 15 wt % of said oil and includes less than 40 wt % of said oil; and/or
the sum of the wt % of all oils and fats in said foodstuff excluding any oil/fat in the fungus is at least 15 wt % and said sum is less than 40 wt %;
wherein said fungus is a filamentous fungus and said fungus comprises filaments having number average lengths of less than 500 μm.

7-8. (canceled)

9. A foodstuff according to claim 1, wherein said fungus is a filamentous fungus which is a Fusarium species, optionally Fusarium venenatum.

10. A foodstuff according to claim 1, wherein said foodstuff includes at least 1.0 wt % of said fungus on a dry mass basis; and optionally said foodstuff includes at least 1.0 wt % of protein on a dry mass basis.

11. (canceled)

12. A foodstuff according to claim 1, wherein, in said foodstuff, the sum of the wt % of said fungus on a dry mass basis and said oil is at least 30 wt % and is less than 50 wt %; and/or
wherein, in said foodstuff, the sum of the wt % of each fungus on a dry mass basis and each oil/fat is at least 30 wt % and said sum is less than 50 wt %.

13. A foodstuff according to claim 12, wherein said foodstuff include at least 5 wt % of water and includes less than 50 wt % water.

14. A foodstuff according to claim 12, wherein, in said foodstuff:
a ratio defined as the weight of said oil divided by the weight of water is greater than 0.2 and the ratio is less than 3.0; and/or
a ratio defined as the total weight of oil(s)/fats(s) divided by the weight of water is greater than 0.2 and the ratio is less than 3.0.

15. A foodstuff according to claim 1, wherein said foodstuff includes:
1 to 30 wt % of said fungus on a dry mass basis; and
15 to 40 wt % of said oil;
wherein said foodstuff includes one or more starches, wherein the sum of the wt % of starches in said foodstuff is at least 4.0 wt % and said sum is less than 30.0 wt %.

16. (canceled)

17. A foodstuff according to claim 1, wherein said foodstuff is a hard vegan cheese and includes at least 1.0 wt % of a starch which has greater than 20.0 wt % of amylose; and/or
wherein said foodstuff is a semi-hard vegan cheese and the total wt % of starch(es) which include(s) less than 10 wt % of amylose is greater than the total wt % of starches in the foodstuff which include greater than 20.0 wt % amylose.

18. (canceled)

19. A foodstuff according to claim 15, wherein, in said foodstuff, the sum of the wt % of each fungus, each oil/fat, each starch and water is at least 70.0 wt % and said sum is less than 99.0 wt %.

20. A foodstuff according to claim 15, wherein said foodstuff includes less than 4.0 wt % (preferably less than 0.5 wt %) of hydrocolloids in total.

21. A foodstuff according to claim 15, wherein said foodstuff includes:
less than 0.1 wt % of hydrocolloids in total; and/or
less than 4.0 wt % (preferably less than 0.5 wt %) of non-starch based carbohydrate polymers in total; and/or
less than 4.0 wt % (preferably less 0.5 wt %) of sucrose; and/or
less than 4.0 wt % (preferably less than 0.5 wt % of lactic acid); and/or
less than 4.0 wt % (preferably less than 0.5 wt %) of lactic acid producing bacteria; and optionally, wherein, in said foodstuff, the sum of the wt % of each fungus, each oil/fat, each starch, each flavourant, salt, and water is at least 80.0 wt %.

22-23. (canceled)

24. A foodstuff according to claim 15, wherein said foodstuff is in the form of a block having a hardness, assessed as described in Test 1, of at least 1000 g and less than 40000 g.

25. A foodstuff according to claim 15, wherein said foodstuff is a semi-hard vegan cheese having a hardness in the range 1000 g to 12000 g.

26. A process for making a foodstuff according to claim 1, the process comprising mixing a fungus and an oil;
wherein said process is for producing a foodstuff having a hardness, assessed as described in Test 1, of at least 1000 g and less than 40000 g.

27. (canceled)

28. A foodstuff which is a vegan cheese, the foodstuff comprising:
a fungus; and
an oil;
wherein said oil is coconut oil;
wherein said foodstuff includes at least 15 wt % of said oil and includes less than 40 wt % of said oil;
wherein the sum of the wt % of all oils and fats in said foodstuff excluding any oil/fat in the fungus is at least 15 wt % and said sum is less than 40 wt %;
wherein said fungus is a filamentous fungus;
wherein said foodstuff includes at least 1.0 wt % of said fungus on a dry mass basis;
wherein said foodstuff includes:
1 to 30 wt % of said fungus on a dry mass basis; and
15 to 40 wt % of said oil.

29. A foodstuff according to claim 28, wherein said foodstuff is a hard vegan cheese and includes at least 2.0 wt % of a starch which has greater than 20.0 wt % of amylose or said foodstuff is a semi-hard vegan cheese having a hardness in the range 1000 g to 12000 g.
